(19) United States
(12) Patent Application Publication
(10) Pub. No.: US 2024/0269115 A1
(43) Pub. Date: Aug. 15, 2024

(54) PYRAZOLONE COMPOUNDS FOR USE IN DEGENERATIVE RETINAL DISEASES

(71) Applicant: FLONEXT S.R.L., Firenze (IT)

(72) Inventors: Pierangelo GEPPETTI, Firenze (IT); Romina NASSINI, Firenze (IT); Francesco DE LOGU, Castiglione della Pescaia (GR) (IT)

(73) Assignee: FLONEXT S.R.L., Firenze (IT)

(21) Appl. No.: 18/567,807

(22) PCT Filed: Jun. 7, 2022
(86) PCT No.: PCT/IB2022/055275

§ 371 Date: Dec. 7, 2023

(30) Foreign Application Priority Data
Jun. 9, 2021 (IT) 102021000015095

(51) Int. Cl. A61K 31/4152; A61K 9/00; A61K 45/06; A61P 27/02; C07K 16/22

(52) U.S. Cl. CPC A61K 31/4152; A61K 9/0048; A61K 45/06; A61P 27/02; C07K 16/22

(57) ABSTRACT

The present invention relates to compounds belonging to the pyrazolone class for use in the prevention and/or treatment of degenerative retinal diseases, in particular in the prevention and/or treatment of macular degeneration. The present invention also relates to a pharmaceutical composition comprising at least one compound belonging to the pyrazolone class, preferably a topical ophthalmic composition, for use in the prevention and/or treatment of degenerative retinal diseases, preferably macular degeneration.

The present invention relates to compounds belonging to the pyrazolone class for use in the prevention and/or treatment of degenerative retinal diseases, in particular in the prevention and/or treatment of macular degeneration.

The present invention also relates to a pharmaceutical composition comprising at least one compound belonging to the pyrazolone class, preferably a topical ophthalmic composition, for use in the prevention and/or treatment of degenerative retinal diseases, preferably of macular degeneration.

### STATE OF THE ART

The retina is the transparent light-sensitive structure located in the ocular fundus. The central area of the retina, called macula, contains numerous photoreceptors, called cones, which are light-sensitive cells responsible for central and colour vision. The peripheral area of the retina, which surrounds the macula, contains photoreceptors, called rods, which respond to lower light levels but are not sensitive to colours. The rods in fact allow vision in low light conditions, but not chromatic perception.

The retina can be affected by different types of pathologies which, depending on the retinal area affected, can have serious repercussions on vision.

Some diseases affecting the retina can be traced back to degenerative diseases and can severely impair vision and lead to blindness.

Macular degeneration is an age-related multifactorial disease affecting the macula. It is a progressive disease and is the main cause of irreversible blindness in adults over the age of 50. This disease has a prevalence ranging from 8.5% to 11% in the 65-74 age group, and 27% over the age of 75. It is therefore a disease linked to ageing and therefore destined to have an ever-wider impact on the world population due to increased life expectancy.

Two different forms of age-related macular degeneration are known: the dry form (non-exudative or atrophic) and the wet form (exudative or neovascular).

All age-related macular degenerations begin as a dry form and about 15% of them then evolve into the wet form.

The dry form of age-related macular degeneration causes changes of the retinal pigment epithelium, typically visible as dark punctiform areas. The retinal pigment epithelium plays a critical role in keeping the rods and cones healthy and well-functioning. The accumulation of waste products from the cones and rods can lead to the formation of drusen, visible as yellow spots that characterize the initial phase of age-related macular degeneration.

The dry form is characterized by a progressive thinning of the central retina, which is poorly nourished by the capillaries and atrophies, leading to the formation of an atrophic lesion in the macular area. Areas of chorioretinal atrophy (referred to as geographic atrophy) occur in more advanced cases of the dry form of age-related macular degeneration. The long-term consequence of this slow degenerative process is the impairment of the functionality of the macula, which is no longer able to collect the light impulses properly. The majority of patients retain sufficient vision capacity for reading and driving. Areas of central blindness (scotomas) usually occur late in the disease and can sometimes become severe. Symptoms are generally bilateral.

Wet macular degeneration, on the other end, is characterized by the growth of abnormal blood vessels from the choroid, in correspondence with the macula (choroidal neovascularization). Focal macular edema or hemorrhage can result in a raised macular area or cause a localized detachment of the retinal pigment epithelium. Finally, untreated neovascularization causes a submacular disciform scar.

In general, wet macular degeneration, more aggressive than the dry form, can cause rapid and severe loss of central vision, caused by scarring of the blood vessels.

Patients with the wet form of age-related macular degeneration show rapid loss of the visual function, usually within days or weeks. The first symptom is generally visual distortion, characterized by the presence of scotomas or metamorphopsia (curvature of straight lines) secondary to the formation of new vessels near or in the center of the macula. These newly formed blood vessels come almost exclusively from the choroid (choroidal neovascularization) and cause the formation of a fibrovascular scar that destroys the central retina. Wet macular degeneration usually affects one eye at a time so the symptoms of macular degeneration are often unilateral.

Although peripheral vision and colour vision are generally not affected, the patient can become legally blind (visual acuity <20/200) on the affected eye side if age-related macular degeneration is left untreated.

Most available treatments aim to prevent or cure the wet form of neovascular macular degeneration.

However, to date there is still no established treatment for the dry form.

Patients with extensive drusen, pigmentation changes, and/or geographic atrophy can reduce their risk of developing advanced age-related macular degeneration by 25% by taking antioxidant and mineral vitamin supplements that generally include at least lutein or other vitamins, and sometimes zinc or other nutrients. Recently, omega-3 fatty acids have been prescribed to patients suffering from the dry form of age-related macular degeneration and included together with antioxidants among the dietary supplements on the market.

Patients with the unilateral wet form of age-related macular degeneration should take the daily nutritional supplements recommended for the dry form in order to reduce the risk of vision loss in the other eye.

Other non-resolving or otherwise invasive types of treatment used for the wet form include, for example, laser-induced thermal photocoagulation, photodynamic therapy, transpupillary thermotherapy, subretinal surgery and macular translocation surgery.

The pharmacological treatment of choice for the wet form of maculopathy includes the periodic administration by intravitreal injection of vascular endothelial growth factor antagonist drugs (anti-VEGF) such as ranibizumab, bevacizumab, or aflibercept. On average, 6 or 7 intravitreal injections are given in the first year of treatment.

Furthermore, corticosteroids, such as triamcinolone, can also be administered together with anti-VEGF drugs, again by intraocular injection.

However, the intravitreal mode of administration adopted by most treatments available today entails several adverse effects. In fact, the intravitreal injection is an invasive route of administration that can lead to increased intraocular pressure, headache, vitritis (inflammation of the eye), vitreous detachment, retinal hemorrhage (bleeding from the back of the eye), visual disturbances and ocular pain. The most dangerous complication is septic endophthalmitis, a serious intraocular inflammatory pathology due to the infection of the vitreous cavity, which, although occurring infrequently (about 1/1000) can lead not only to pain and redness, but also to much more serious consequences such as loss of vision up to total blindness. It should also be remembered that the risk can be cumulative in the case of repeated injections.

It is therefore evident that for the long-term treatment of age-related macular degeneration it is necessary to provide new, non-invasive therapies, which allow prevention and/or prolonged treatment without incurring side effects related to the method of administration.

### SUMMARY OF THE INVENTION

The Applicant addressed the problem of providing a new therapy for the prevention and/or prolonged treatment of degenerative pathologies of the retina, in particular macular degeneration, which does not involve the drawbacks and side effects of current therapies, in particular therapies requiring intravitreal administration, and possibly of equal or greater efficacy.

The Applicant has surprisingly found that a new therapy based on the administration of compounds belonging to the pyrazolone class can be useful in the prevention and treatment of degenerative pathologies of the retina, such as macular degeneration.

Therefore, a first aspect of the present invention is a compound belonging to the pyrazolone class for use in the prevention and/or treatment of at least one degenerative retinal disease selected from macular degeneration, diabetic retinopathy, retinal detachment, central serous chorioretinopathy, hypertensive retinopathy, macular hole, macular pucker and myodesopsia (floaters) and myopic maculopathy.

Advantageously, the Applicant has found that, among the compounds belonging to the pyrazolone class, dipyrone and propyphenazone are particularly effective in the prevention and/or treatment of macular degeneration.

In particular, the Applicant has observed that the compounds belonging to the pyrazolone class, preferably dipyrone and propyphenazone, make it possible to prevent and/or treat both forms of macular degeneration, namely dry senile macular degeneration and wet senile macular degeneration.

Another aspect of the present invention is a pharmaceutical composition comprising at least one compound belonging to the pyrazolone class and at least one pharmaceutically acceptable excipient for use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy, preferably macular degeneration.

Preferably, the pharmaceutical composition according to the invention is a topical ocular ophthalmic composition.

A further aspect of the present invention is a kit comprising the topical ocular ophthalmic composition, a container containing it and a dispenser, wherein said composition is for use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy, preferably macular degeneration.

Preferably, the topical ophthalmic composition within said kit is an aqueous solution.

Another aspect of the present invention is a method of prevention and/or treatment of a degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy which comprises administering to a patient at least one compound belonging to the pyrazolone class and/or an ophthalmic composition comprising at least one compound belonging to the pyrazolone class for use according to the invention.

The last aspect of the present invention is a combination of at least one compound belonging to the pyrazolone class and an anti-VEGF drug and/or a corticosteroid drug for simultaneous, separate or sequential use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy, preferably macular degeneration in both its forms.

### DETAILED DESCRIPTION OF THE INVENTION

One aspect of the present invention is a compound belonging to the pyrazolone class for use in the prevention and/or treatment of at least one degenerative retinal disease. In particular, the compounds according to the invention have proved useful in the prevention and/or treatment of macular degeneration.

The pyrazolones for use according to the present invention are pyrazole-derived compounds represented by the following general formula (1):

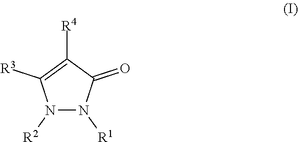

wherein R1 and R2 are independently selected from H, linear or branched C1-C6 alkyl, aryl optionally substituted with OH, C1-C6 alkoxy, linear or branched C1-C6 alkyl or halogen; R3 is selected from linear or branched C1-C6 alkyl and OH; R4 is selected from H, linear or branched C1-C8 alkyl, linear or branched C2-C8 alkenyl, a group—(CH₂)₁₋₄—CO-linear or branched C1-C6 alkyl, an amino group mono or disubstituted with linear or branched C1-C6 alkyl or with a group —(CH₂)₍₁₋₃₎—SO₃H and combinations thereof, a —NHCO-aryl or —NHCO-heteroaryl group, and pharmaceutically acceptable salts thereof.

In the present description, the term alkylene indicates a hydrocarbon chain having at least one carbon-carbon double bond.

Preferred pyrazolones for use according to the present invention are the compounds of formula (1) wherein R1 and R2 are independently selected from H, linear or branched C1-C3 alkyl, phenyl optionally substituted with OH; R3 is selected from linear or branched C1-C3 alkyl and OH; R4 is selected from H, linear or branched C1-C6 alkyl, linear or branched C2-C6 alkenyl, a group -(CH₂)₍₂₋₃₎—CO-linear or branched C1-C4 alkyl, an amino group mono or disubstituted with linear or branched C1-C3 alkyl, with a group —(CH₂)₍₁₋₂₎—SO₃H and combinations thereof, a —NHCO-nitrogenated heteroaryl group, and pharmaceutically acceptable salts thereof.

In a preferred embodiment, the pyrazolones for use according to the present invention are the compounds of formula (1) wherein R1 and R2 are independently selected from H, linear or branched C1-C3 alkyl, phenyl optionally substituted with OH; R3 is a linear or branched C1-C3 alkyl; R4 is selected from H, linear or branched C1-C6 alkyl, linear or branched C2-C6 alkenyl, a group -(CH₂)(₂₃)—CO-linear or branched C1-C4 alkyl, an amino group mono or disubstituted with linear or branched C1-C3 alkyl, with a group —(CH₂)₍₁₋₂₎—SO₃H and combinations thereof, a —NHCO-nitrogenated heteroaryl group, and pharmaceutically acceptable salts thereof.

Examples of pharmaceutically acceptable salts are the salts obtained by adding an acid or a base to a pyrazolone of formula (1), capable of forming acid or basic salts with their basic groups, for example, amine or sulphonic or ketoenolic acids and the like.

Pharmacologically acceptable acid addition salts can be formed with inorganic acids, for example hydrochloric acid, hydrobromic acid, sulfuric acid, nitric acid, phosphoric acid and the like, or with organic acids such as for example acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulphosalicylic acid and the like.

Pharmaceutically acceptable addition salts of bases can be formed with inorganic bases, such as ammonium salts and salts of the metals of columns I to XII of the periodic table pharmaceutically acceptable such as sodium, potassium, calcium, magnesium, iron, silver, zinc and copper, or with organic bases, for example, primary, secondary and tertiary amines, substituted amines including natural substituted amines or cyclic amines, and the like.

Examples of suitable organic amines include isopropylamine, choline, diethanolamine, diethylamine, lysine, meglumine, piperazine and tromethamine.

Even more preferred pyrazolones for use according to the present invention are the compounds of formula (1) reported in Table 1 below:

TABLE 1

| NAME | R1 | R2 | R3 | R4 |
|---|---|---|---|---|
| Aminophenazone or | Phenyl | CH₃ | CH₃ | —N(CH₃)₂ |
| pyramidon | | | | |
| Dipyrone or sodium | Phenyl | CH₃ | CH₃ | —N(CH₃)—CH₂SO₃⁻Na⁺ |
| metamizolo | | | | |
| Phenazone | Phenyl | CH₃ | CH₃ | H |
| Propyphenazone or | Phenyl | CH₃ | CH₃ | CH(CH₃)₂ |
| isopropyl-antipyrine | | | | |
| Nifenazone | Phenyl | CH₃ | CH₃ | —NHCO—3-pyridyl |
| Phenylbutazone | Phenyl | Phenyl | OH | —(CH₂)₃—CH₃ |
| Pyrasanone or | Phenyl | Phenyl | OH | —(CH₂)₃—CH₃ |
| phenylbutazone | | | | |
| piperazine salt | | | | |
| Oxyphenylbutazone | 4-OH- | Phenyl | OH | —(CH₂)₃—CH₃ |
| | phenyl | | | |
| Kebuzone or | Phenyl | Phenyl | OH | —(CH₂)₂—CO—CH₃ |
| ketophenylbutazone | | | | |
| Feprazone o metrazone | Phenyl | Phenyl | OH | —CH₂—CH═C(CH₃)₂ |
| Mofebutazone or | H | Phenyl | OH | —(CH₂)₃—CH₃ |
| monophenylbutazone | | | | |
| Tribuzone or trimetazone | Phenyl | Phenyl | OH | —(CH₂)₂—CO—t-Bu |

Drugs belonging to the pyrazolone class have been used since the 1950s in the treatment of ankylosing spondylitis, in acute gout and in musculoskeletal pathologies of various kinds due to their non-steroidal anti-inflammatory (NSAID), antipyretic and analgesic activity.

The compound according to the invention belonging to the pyrazolone class is preferably selected from aminophenazone, dipyrone, phenazone, propyphenazone, nifenazone, phenylbutazone, pyrasanone, oxyphenylbutazone, kebuzone, feprazone, mofebutazone, tribuzone and mixtures thereof. In a particularly preferred embodiment, the compound belonging to the pyrazolone class is selected from dipyrone, propyphenazone and mixtures thereof.

In an even more preferred embodiment, the compound belonging to the pyrazolone class is selected from dipyrone, propyphenazone and mixtures thereof, and said degenerative retinal disease is macular degeneration.

In one embodiment, said macular degeneration is dry senile macular degeneration.

In another embodiment, said macular degeneration is wet senile macular degeneration.

Dipyrone, also known as metamizole (trade name Novalgina®), is a non-steroidal analgesic drug having the following chemical formula:

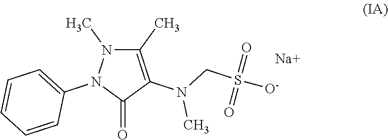 [SMILES: Cc1c(N(C)CS(=O)(=O)[O-])c(=O)n(-c2ccccc2)n1C ; [Na+]]

Dipyrone is commonly used as an antipyretic and painkiller against headaches, fever, dental pain, menstrual pain and others.

Propyphenazone, also known as isopropylantipyrine, which has long been marketed in combination with other active ingredients under the names, for example, of Optalidon® or Saridon®, is a phenazone derivative with similar analgesic and antipyretic effects and the following chemical formula:

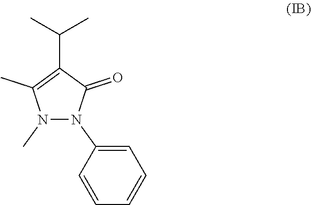 [SMILES: Cc1c(C(C)C)c(=O)n(-c2ccccc2)n1C]

The Applicant has advantageously observed that pyrazolones, in particular dipyrone and propyphenazone, but not a classic cyclooxygenase (COX) inhibitor such as Indomethacin, are able to reduce the degenerative retinal damage (see experimental section).

The compounds according to the invention, preferably selected from dipyrone and/or propyphenazone, can prevent and/or treat at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy. The particularly preferred compounds according to the invention, i.e. dipyrone and/or propyphenazone, can be effective in the prevention and treatment of macular degeneration in both its forms (dry senile macular degeneration and wet senile macular degeneration).

According to a preferred aspect of the invention, the compounds of the invention, preferably dipyrone and/or propyphenazone, can prevent and/or treat a particular type of degenerative retinal disease: macular degeneration, in both its forms (dry senile macular degeneration and wet senile macular degeneration). Therefore, in a particularly preferred embodiment, the degenerative retinal disease that the compounds according to the invention can treat is macular degeneration, in both its forms. Macular degeneration falls into the macro category of maculopathies. In the present invention, the term “macular degeneration” is used to indicate a particular type of degenerative maculopathy.

The term “maculopathy” refers to a pathology that affects the central part of the retina, called macula. Maculopathies can be classified into acquired maculopathies, myopic maculopathies and hereditary maculopathies.

According to another aspect of the invention, compounds belonging to the pyrazolone class, preferably selected from dipyrone and/or propyphenazone, can be used for the prevention and/or treatment of myopic maculopathy.

Myopic maculopathy occurs in people with degenerative or pathological myopia. In particular, in subjects suffering from myopic maculopathy, the retina is unable to adapt to the elongation of the bulb and suffers injuries.

In pathological myopia, macular hemorrhages can occur with sudden decrease in visual acuity, sometimes with image distortion.

The most frequent acquired maculopathy is age-related macular degeneration.

Macular degeneration is a disease characterized by the deterioration of the macula, the central portion of the retina responsible for central vision.

This pathology is often referred to as age-related macular degeneration or senile macular degeneration as it occurs mainly in individuals over the age of 60. In fact, many elderly people develop the condition as part of the natural aging process.

In the present invention, the expressions “age-related macular degeneration” and “senile macular degeneration” both indicate the degenerative maculopathy of the retina described above.

According to the present invention, the compound belonging to the pyrazolone class can be useful for the prevention and/or treatment of both forms of macular degeneration, i.e. dry senile macular degeneration and/or wet senile macular degeneration.

In fact, the compounds of the invention, in particular dipyrone and propyphenazone, have shown efficacy in reducing the oxidative damage of the retinal epithelium caused by the injection of sodium iodate (NalO₃) in a mouse model representative of both forms of macular degeneration (see Example 3). The mouse model of macular degeneration on which the experiments were carried out in order to test the compounds according to the invention was obtained by intravenous administration of NalO₃. NalO₃, metabolite of Septojod (an old drug no longer used to treat septicemia), was identified as being responsible for the blindness observed in patients treated with the precursor drug. Subsequently it was shown that sodium iodate selectively damages the cells of the retinal pigment epithelium (RPE, from retinal pigment epithelium), a layer of pigmented cells that lies above the choroid and nourishes the visual cells of the retina, promoting in these cells necroptosis phenomena that spread from the central part of the retina to more peripheral areas (patchy RPE degeneration).

It has been observed that the damage from NalO₃ can subsequently spread also to the photoreceptors, which degenerate and die due to a mechanism of apoptosis. Based on these data, NalO₃ is an important tool for the study of diseases such as age-related macular degeneration (AMD). In fact, it has been observed that even if administered systemically, NalO₃ exerts a specific toxic action on the retinal pigment epithelium cells and on the photoreceptors through the production of oxidative stress.

Over the years, the animal model of macular degeneration obtained by injection of NalO₃ has been validated through a series of scientific publications which have made it possible to establish that in animals treated with NalO₃ damage is observed at the central pole of the retina and that, at higher doses of NalO₃, it can also extend to the peripheral area (Kiuchi, Current Eye Research 2002; Machalinska, Neurochemical Res. 2010; Wang, Invest Ophthalmol Vis Sci 2014; Commentaries Neural Regeneration Research 2014; Hanus, Cell Death Disc 2016; Chowers, Invest Ophtalmol 2017 and Koh, Journal of Photochemistry & Photobiology, 2019).

It was also observed that the pathological phenomenon produced in mice treated with NalO₃ presents the characteristics observed in patients suffering from acquired degenerative disorders, such as age-related macular degeneration and toxic retinopathies due to intoxication by drugs such as chloroquine, thioridazine or chlorpromazine.

In particular, according to Hanus (Cell Death Disc 2016), retinal degeneration induced by NalO₃ in animal models shows at least two characteristics which are similar to age-related macular degeneration affecting humans. First, it was observed that low doses lead to an irregular loss of cells in the RPE layer. Second, it was observed that the loss of cells of the RPE layer affects not only the photoreceptors, but also the underlying choriocapillaris layer.

In detail, morphological examination of the retina after the administration of 100 mg/kg of NalO₃ in mice shows depigmentation, swelling and vacuolization, suggesting necrosis of the RPE layer. Furthermore, in the same work it was observed that higher dosages of NalO₃ cause increased damage to the RPE layer which, over time, became thinner and thinner, and also damage to the photoreceptors, while dosages lower than 10 mg/kg have proven to have little effect on the retina.

A more recent study (Koh, Journal of Photochemistry & Photobiology, 2019) confirmed that in this model the specific toxicity for RPE caused by Na1O₃ recapitulates the late effects of age-related macular degeneration and retinitis pigmentosa in humans.

To summarize, the histopathological changes caused by NalO₃ in the experimental animal typical of macular degeneration are: discontinuity of the RPE layer, damage to photoreceptors and infiltration of macrophages (tissue mononuclear cells capable of engulfing foreign or damaged cells or materials and destroying them).

The model presents all the histopathological features observed in humans and, therefore, is a valid model of macular degeneration.

A further aspect of the present invention relates to a pharmaceutical composition, preferably an ophthalmic composition, more preferably a topical ocular ophthalmic composition, comprising a therapeutically effective amount of at least one compound belonging to the pyrazolone class and at least one pharmaceutically acceptable excipient for use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy.

In the pharmaceutical composition for use according to the invention, said at least one compound belonging to the pyrazolone class of formula (1) is selected from aminophenazone, dipyrone, phenazone, propyphenazone, nifenazone, phenylbutazone, pyrasanone, oxyphenylbutazone, kebuzone, feprazone, mofebutazone, tribuzone and mixtures thereof.

In a preferred embodiment, the pharmaceutical composition, preferably ophthalmic, according to the invention comprises dipyrone and/or propyphenazone as compounds belonging to the pyrazolone class.

In a particularly preferred embodiment, the pharmaceutical composition, preferably ophthalmic, comprising dipyrone and/or propyphenazone as preferred compounds is used for the prevention and/or treatment of macular degeneration, in particular dry age-related macular degeneration or wet age-related macular degeneration.

According to an aspect, the ophthalmic composition comprises a plurality of compounds belonging to the pyrazolone class, preferably at least two compounds, said compounds belonging to the pyrazolone class being preferably selected from dipyrone and propyphenazone.

The pharmaceutical composition according to the invention, preferably ophthalmic, preferably comprising dipyrone and/or propyphenazone as compounds belonging to the pyrazolone class, can be advantageously used for the prevention and/or treatment of degenerative retinal diseases selected from macular degeneration, diabetic retinopathy, retinal detachment, central serous chorioretinopathy, hypertensive retinopathy, macular hole, macular pucker, myodesopsia (floaters) and myopic maculopathy.

Preferably, said retinal degenerative disease is macular degeneration, in particular dry age-related macular degeneration and wet age-related macular degeneration.

The pharmaceutical composition according to the invention can be administered according to any route of administration, systemic or local, as long as it is suitable for achieving concentrations at the retinal level that are effective for the prevention or treatment of the pathology in question.

Preferably, the pharmaceutical composition according to the invention is an ophthalmic composition, suitable for being administered internally or externally to the eye.

According to an embodiment, the present composition is a composition suitable for administration to the posterior segment of the eye, for example by injection or surgical implant, in particular suitable for administration to the retina, the sclera, the posterior chamber, the vitreous chamber, the subretinal space or to the suprachoroidal segment of the eye.

According to another embodiment, the present composition is a composition suitable for administration to the anterior segment of the eye, by injection or by surgical implant.

In another more preferred embodiment, the composition according to the invention is a topical ophthalmic composition, suitable to be administered externally to the eye, for example by application in the lower eyelid pouch or conjunctival fornix, on the outer surface of the cornea or of the sclera.

The topical ophthalmic composition according to the invention can be formulated, for example, in the form of a solution, suspension, emulsion, gel, ointment, ocular insert or therapeutic contact lens. The topical use of the composition of the invention, for example in the form of drops or eye drops, advantageously allows treating in a non-invasive way one or more retinal diseases, preferably macular degeneration, and to avoid the inconveniences and side effects of intravitreal administration, today commonly used for the treatment of macular degeneration.

Furthermore, since generally only a small fraction of the drug dose administered topically is effectively absorbed, it follows that the possible systemic side effects of the compounds belonging to the pyrazolone class according to the invention are expected to be minimal.

The ophthalmic composition according to the invention can comprise one or more ophthalmologically acceptable additives and/or excipients selected from those commonly used for the ophthalmic formulations.

An “ophthalmologically acceptable excipient” is an inert excipient which allows the administration of a medicament to the eye and/or eyelids, to treat an ocular disease or condition without exerting deleterious effects on the eye. Generally, these are substances that contribute to increasing the efficacy and tolerability of the products in which they are contained, as well as favoring their conservation over time.

Examples of said ophthalmologically acceptable additives or excipients are viscosifiers, permeation enhancers, buffering agents, osmolarity regulators, antioxidants, preservatives and surfactants.

Viscosifiers, which have the function of increasing the viscosity of the composition and consequently the contact time of the drug with the ocular surfaces, are preferably selected from cellulose derivatives, preferably hydroxymethyl cellulose, hydroxyethyl cellulose, hydroxypropyl methylcellulose, methylcellulose; polyethylene glycol, polyvinylpyrrolidone, polyvinylacetic alcohol, dextran, gelatin, glycerin, polysorbate 80 and other gelling agents.

Permeation enhancers, which have the function of increasing the permeability of the drug across ocular membranes, are preferably selected from cyclodextrins, chelating agents, corona ethers, bile acids and bile salts.

Buffering agents have the function of providing and maintaining the pH of the composition as close as possible to the physiological pH, preferably between 6 and 8. This action is essential to allow good tolerability of the preparations and to maintain their efficacy. The preferred buffer is phosphate buffer, but other buffers capable of maintaining the pH within the desired range are also included, as long as they are suitable for ophthalmic use.

Osmolarity regulators are salts capable of making the liquid composition isotonic with ocular fluids. The preferred salt is sodium chloride (NaCl), but other biologically acceptable salts can be used, such as potassium chloride (KCl), calcium chloride (CaCl₂) and magnesium chloride (MgCl₂) and mixtures thereof, or substances such as propylene glycol, glycerin, dextrose, dextran 40 and 70 or the buffer substances described above.

Antioxidant agents prevent or delay the deterioration of products resulting from the action of atmospheric oxygen. Among these substances, the most commonly used are ethylenediaminetetraacetic acid (EDTA), thiourea, sodium thiosulfate, sodium metabisulphite and sodium bisulfite.

Preservatives are substances that inhibit bacterial proliferation that can occur after opening the product. Suitable preservatives include for example quaternary ammonium compounds such as benzalkonium chloride, cetyltrimethylammonium bromide and cetylpyridinium chloride, benzethonium hydrochloride, chlorobutanol, EDTA, mercury preservatives (such as thimerosal), phenylethyl alcohol, sodium benzoate, sodium propionate and sorbic acid.

Many of these agents are surfactant compounds which, in addition to inhibiting bacterial proliferation, favor the penetration of drugs through the cornea.

Surfactants have the function of making the composition stable and favoring the penetration of the active ingredients into the ocular structures. Examples of surfactants are polysorbates and poloxamers.

In one embodiment, the ophthalmic composition for use according to the invention is an aqueous ophthalmic composition, for example in the form of eye drops for topical administration to the anterior segment of the eye. The aqueous ophthalmic composition according to said embodiment comprises water in an amount sufficient to achieve the appropriate concentration of the components of the composition.

Preferably, in the liquid, preferably aqueous, ophthalmic composition, the compound belonging to the pyrazolone class can be present in concentrations ranging from about 0.0001% to about 5% w/v, more preferably from about 0.01% to about 1% w/v, even more preferably from about 0.1% to about 1% w/v of the aqueous composition.

An ophthalmic composition for use according to the invention can for example comprise a therapeutically effective amount of at least one compound belonging to the pyrazolone class, sodium chloride, magnesium chloride, mono and di-basic sodium phosphate and water for ophthalmic use.

In one embodiment, the topical ophthalmic composition, preferably a liquid composition, for use according to the invention can be part of a kit comprising the composition, a container containing it and a dispenser.

In particular, said kit comprises an ocular topical ophthalmic composition as described above, a container that contains it and a dispenser, wherein said composition is for use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy, preferably macular degeneration (both dry age-related macular degeneration and wet age-related macular degeneration).

Preferably, the topical ophthalmic composition within said kit is an aqueous solution.

In the case of eye drops, the dispenser is a drop dispenser.

In another embodiment, the pharmaceutical composition, preferably ophthalmic, for use according to the invention can further comprise at least one other pharmaceutically active compound.

In a preferred embodiment, the pharmaceutical composition, preferably ophthalmic, for use according to the invention can further comprise one or more antagonist drugs of vascular endothelial growth factor (anti-VEGF) and/or corticosteroid drugs.

A further aspect of the present description also relates to a method for the prevention and/or treatment of at least one degenerative retinal disease, preferably selected from macular degeneration, diabetic retinopathy, retinal detachment, central serous chorioretinopathy, hypertensive retinopathy, macular hole, macular pucker, myodesopsia (floaters) and myopic maculopathy which includes the administration to a subject of at least one compound belonging to the pyrazolone class of formula (I) and/or of a pharmaceutical composition, preferably ophthalmic, comprising at least one compound belonging to the pyrazolone class, and one or more pharmaceutically acceptable excipients.

In a preferred embodiment, said degenerative retinal disease is macular degeneration and said compound belonging to the pyrazolone class is selected from dipyrone, propyphenazone and mixtures thereof.

The doses used ocularly in experiments carried out on the validated mouse model of macular degeneration (Examples 1-3) correspond to approximately 40 mg of dipyrone and 28 mg of propyphenazone in a 70 kg individual.

Accordingly, the method according to the invention can indicatively comprise the topical ocular administration of 1-80 mg/per administration of at least one compound belonging to the pyrazolone class, for a daily total, for example, of 1-5 administrations. The actual dose and regimen for administering the compound for use according to the invention in the treatment or prevention of the aforementioned diseases depend on many factors, such as the route of administration or the degree of distress of the individual who receives the treatment.

These doses can generally be much lower than the doses used for analgesic purposes for systemic administration in humans (500-1000 mg for dipyrone and 125-286 mg for propyphenazone) with undoubted advantages in terms of reduced systemic side effects.

In an alternative embodiment, the method comprises administering one or more drugs commonly in use for the treatment of degenerative retinal diseases, preferably macular degeneration, in combination with the compound belonging to the pyrazolone class, or with the ophthalmic composition for use according to the invention.

Said drugs commonly used for the treatment of degenerative retinal diseases are selected from antagonist drugs of vascular endothelial growth factor (anti-VEGF) and/or corticosteroid drugs.

In a particularly preferred embodiment, the method comprises administering dipyrone and/or propyphenazone as compounds belonging to the pyrazolone class and/or a composition comprising dipyrone and/or propyphenazone together with drugs commonly used for the treatment of degenerative retinal diseases, preferably antagonist drugs of vascular endothelial growth factor (anti-VEGF) and/or corticosteroid drugs.

In this embodiment, said drug currently in use for the treatment of macular degeneration, preferably an anti-VEGF drug and/or a corticosteroid drug, can be administered before, during or after the administration of the compound belonging to the pyrazolone class and/or of the ophthalmic composition described above.

Examples of anti-VEGF drugs currently in use for the treatment of macular degeneration that can be administered in combination with the ophthalmic composition comprising at least one compound belonging to the pyrazolone class are ranibizumab, bevacizumab, or aflibercept.

Examples of corticosteroid drugs currently in use for the treatment of macular degeneration that can be administered in combination with the ophthalmic composition comprising at least one compound belonging to the pyrazolone class are cortisone drugs, for example selected from cortisone, prednisone, prednisolone, methylprednisolone, meprednisone, beclomethasone, triamcinolone, paramethasone, mometasone, budesonide, fluocinonide, halcinonide, flumethasone, flunisolide, fluticasone, betamethasone, dexamethasone, hydrocortisone and fluocortolone.

The method of prevention and/or treatment of a degenerative retinal disease comprises the administration of at least one of the corticosteroid drugs listed above, preferably when said degenerative retinal disease is selected from diabetic retinopathy, retinal detachment, central serous chorioretinopathy, hypertensive retinopathy.

In another embodiment, the method for the prevention and/or treatment of at least one degenerative retinal disease as defined above, comprises administering to a subject a composition comprising at least one compound belonging to the pyrazolone class and at least one drug selected from anti-VEGF drugs and/or corticosteroid drugs and/or other drugs with different mechanisms of action which are potentially effective in the prevention and treatment of degenerative maculopathy such as GT005, an experimental drug based on gene therapy and able to modulate the activity of the complement system.

In this embodiment, the drug currently in use for the treatment of retinal diseases, preferably macular degeneration, is included in the composition of the invention and is therefore co-administered with the compound belonging to the pyrazolone class and/or the composition comprising said compound.

In a final embodiment, the method comprises administering one or more vitamin supplements commonly in use for the treatment of macular degeneration, in combination with the compound belonging to the pyrazolone class or with the ophthalmic composition for use according to the invention.

In particular, the supplements that can be administered according to the method described above are antioxidant and mineral supplements which generally include at least vitamin C and vitamin E, lutein and zeaxanthin (two carotenoids) and polyphenols and sometimes zinc or other nutrients. Recently, omega-3 fatty acids have been included along with antioxidants among the commercial dietary supplements prescribed to patients suffering from the dry form of age-related macular degeneration.

A final aspect of the present invention relates to a combination of at least one compound belonging to the pyrazolone class and an anti-VEGF drug and/or a corticosteroid drug for simultaneous, separate or sequential use in the prevention and/or treatment of at least one degenerative retinal disease selected from macular degeneration, diabetic retinopathy, retinal detachment, central serous chorioretinopathy, hypertensive retinopathy, macular hole, macular pucker, myodesopsia (floaters) and myopic maculopathy, preferably macular degeneration in both its forms.

Preferably the combination for use according to the invention comprises dipyrone and/or propyphenazone as compounds belonging to the pyrazolone class.

In a preferred embodiment, the combination of at least one compound belonging to the pyrazolone class and an anti-VEGF drug and/or a corticosteroid drug is used for the prevention and/or treatment of macular degeneration, in particular dry age-related macular degeneration and wet age-related macular degeneration.

In said embodiment, the combination comprises an anti-VEGF drug selected from ranibizumab, bevacizumab, or aflibercept.

In said embodiment, the combination comprises a corticosteroid drug selected from cortisone, prednisone, prednisolone, methylprednisolone, meprednisone, beclomethasone, triamcinolone, paramethasone, mometasone, budesonide, fluocinonide, halcinonide, flumethasone, flunisolide, fluticasone, betamethasone, dexamethasone, hydrocortisone and fluocortolone.

In a particularly preferred embodiment of the combination for use in the prevention and/or treatment of macular degeneration (both dry age-related macular degeneration and wet age-related macular degeneration) the compounds belonging to the pyrazolone class are dipyrone, propyphenazone or mixtures thereof.

### DESCRIPTION OF THE FIGURES

FIG. 1A shows immunofluorescence images of the RPE layer in the retina taken on day 4 from NalO₃-induced macular degeneration model mice, after the treatment with NalO₃ or its vehicle (V1) and treated with dipyrone (FN-001), propyphenazone (FN-002) or indomethacin (Ind) or their vehicle (V2). RPE65 indicates the antibody used for staining the RPE layer.

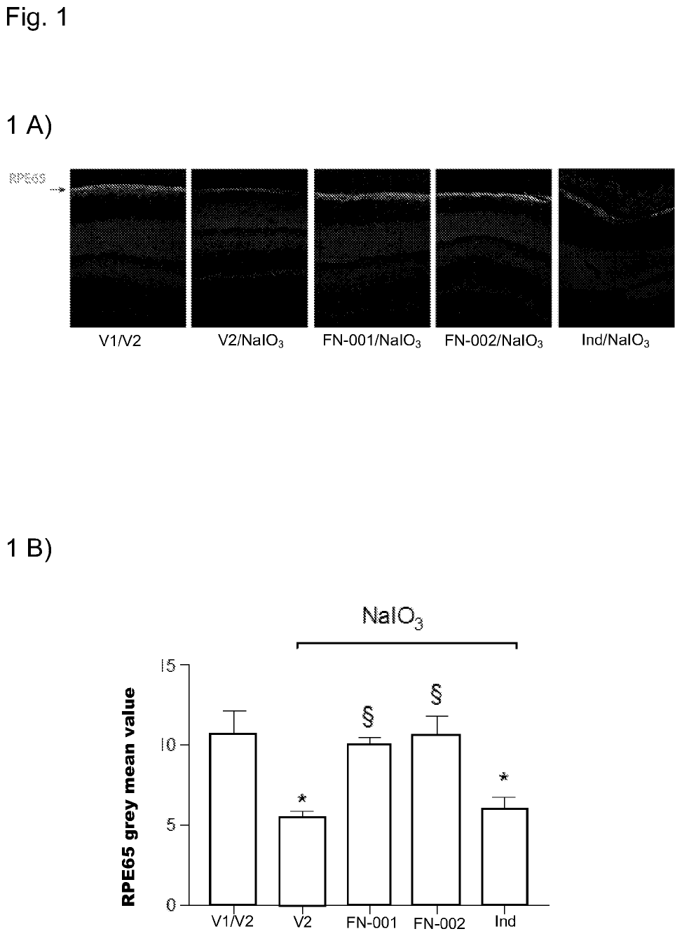

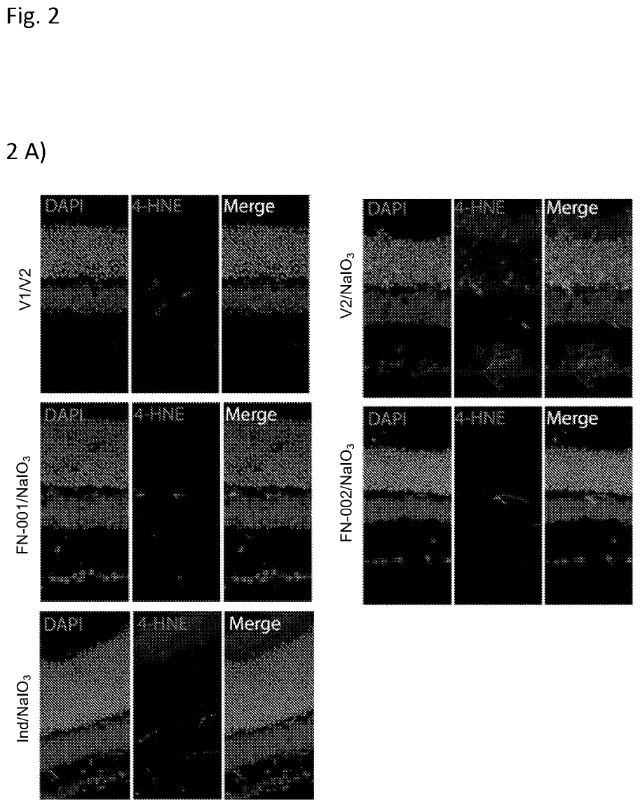

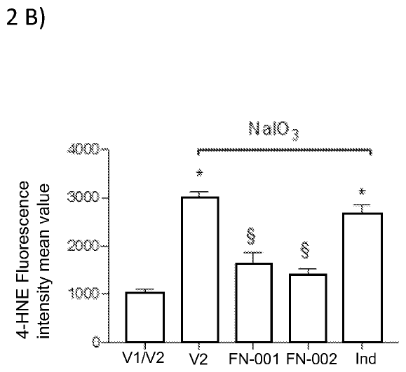

The writings shown on each panel of FIG. 1A indicate: V1/V2: the mice were given the vehicle wherein NalO₃ is dissolved (V1) and before and after they were administered the vehicle wherein the various drugs are dissolved (V2). The V2 vehicle wherein the various drugs were dissolved consisted of 4% dimethyl sulfoxide (DMSO), 4% Tween 80 in 0.9% NaCl and was used as a control. These mice constituted the controls.

V2/NalO₃: the mice were administered the vehicle wherein the various drugs are dissolved (V2) before and after the administration of NalO₃dissolved in its vehicle (V1) to obtain mice with macular degeneration.

FN-001/NalO₃: the mice were treated with dipyrone (FN-001) before and after the administration of NalO₃ dissolved in its vehicle.

FN-002/NalO₃: the mice were treated with propyphenazone (FN-002) before and after the administration of NalO₃ dissolved in its vehicle.

Ind/NalO₃: the mice were treated with indomethacin (Ind) before and after the administration of NalO₃ dissolved in its vehicle.

FIG. 1B is a histogram representing the cumulative data of the immunofluorescence experiment shown in FIG. 1A.

FIG. 2A shows immunofluorescence images of the oxidative stress biomarker 4-hydroxynonenal (4-HNE) in the retina collected on day 4 from mice in the model of NalO₃-induced macular degeneration, after the treatment with NalO₃ or its vehicle (V1) and treated with dipyrone (FN-001), propyphenazone (FN-002) or indomethacin (Ind) or their vehicle (V2). In FIG. 2A, “DAPI” (4′,6-diamidine-2-phenylindole) indicates the organic dye used to mark the nucleus of the retinal epithelium cells, “4-HNE” indicates the reactive species against which the primary antibody to which a second fluorophore-labelled antibody binds and “MERGE” indicates the superposition of the two stains, i.e. the stain obtained with DAPI and the one obtained with the antibodies recognising 4-HNE.

The writings shown on each panel of FIG. 2A indicate: V1/V2: the mice were administered the vehicle wherein the various drugs are dissolved (V1) and before and after they received the administration of the vehicle (V2) wherein NalO₃ is dissolved. These mice constitute the controls.

V2/NalO₃: the mice were administered the vehicle wherein the various drugs are dissolved (V2) before and after the administration of NalO₃ dissolved in its vehicle to obtain mice with macular degeneration.

FN-001/NalO₃: the mice were treated with dipyrone (FN-001) before and after the administration of NalO₃ dissolved in its vehicle.

FN-002/NalO₃: the mice were treated with propyphenazone (FN-002) before and after the administration of NalO₃ dissolved in its vehicle.

Ind/NalO₃: the mice were treated with indomethacin (Ind) before and after the administration of NalO₃ dissolved in its vehicle.

FIG. 2B is a histogram representing the cumulative data of the immunofluorescence experiment shown in FIG. 2A.

### Examples

### Example 1—Mouse Model of Macular Degeneration

In order to test the compounds for use according to the invention, a mouse model of macular degeneration was obtained.

The in vivo experiments were performed in compliance with the Italian legislation (Legislative Decree 26/2014) and the guidelines provided for by the European regulation (EU Directive 2010/63/EU). The study was conducted after the approval of the protocol by the Ministry of Health (protocol no. 687/2020-PR).

To create a valid model of macular degeneration, systemic administration (via the retro-orbital vein) of NalO₃ was carried out in C57BL/6J male mice aged 5-8 weeks and weighing 22-25 g supplied by the Charles River company (Milan, Italy). A total of 30 mice were used to carry out the experiments described below. The animals were kept in a temperature and humidity-controlled environment (12-hour dark/light cycle, free access to food and water).

The experiments were performed in a temperature-controlled room (20 to 22° C.) between 8:00 and 20:00. At the end of the experiment, the animals were euthanized by inhalation of a mixture of 50% O₂/50% CO₂for 1 minute. Unless otherwise noted, the compounds used in the study were all purchased from Merck Life Science SRL (Milan, Italy).

As described above, the mouse model obtained by systemic administration (via the retro-orbital vein) of NalO₃ is a model of macular degeneration. In fact, 3 days after administration, NalO₃ induced in mice persistent retinal damage with characteristics similar to those observed in age-related macular degeneration in humans.

It was observed that the administration of NalO₃ in mice caused damage to the RPE layer (FIG. 1A) and an increase in oxidative stress at the retinal level (FIG. 2A) evidenced by the increased staining of 4-hydroxynonenal (4-HNE), a reactive carbonyl species used as a final indicator of oxidative stress.

### Example 2—Administration of Diovrone, Dropvphenazone, Indomethacin or Vehicle

In order to evaluate the efficacy of dipyrone and propyphenazone, preferred compounds belonging to the pyrazolone class, in reducing and treating the retinal damage typical of macular degeneration, a model of macular degeneration was developed with an experiment on 5 groups of mice. The mice were administered dipyrone, propyphenazone, indomethacin or their vehicle, locally via eye drops.

Indomethacin, a cyclooxygenase (COX) inhibitor drug, was used as a comparison to the effect caused by the administration of dipyrone and propyphenazone.

The drug vehicle consisted of 4% dimethyl sulfoxide (DMSO), 4% Tween 80 in 0.9% NaCl and was used as a control.

In particular, a group of 6 mice (used as a control) were given vehicle (V1) (NaCl, 0.9%) of NalO₃ (1 ml/kg) 60 minutes before the injection into the retro-orbital vein and subsequently given eye drops (5 μl) containing the vehicle (V2) (4% DMSO, 4% Tween 80 in 0.9% NaCl) of the drugs 3 times a day by instillation. Another 24 mice received, in groups of 6 mice each, 60 minutes before the injection into the retro-orbital vein, (1 ml/kg) of NalO₃ (1%, 20 mg/kg) and subsequently by ocular instillation (5 μl) dipyrone (16.65 μg), propyphenazone (11.5 μg), indomethacin (17.85 μg) or their vehicle (4% DMSO, 4% Tween 80 in 0.9% NaCl) 3 times a day.

For each group of mice, the first (day 1) administration of dipyrone, propyphenazone, indomethacin or vehicle (V2) was given 60 minutes before the injection of NalO₃ or its vehicle (V1) and the second and third administration were given 6 and 12 hours after the injection of vehicle (V1) or NalO₃ respectively. In the two days (day 2 and day 3) following the injection of vehicle (V1) or NalO₃, dipyrone, propyphenazone, indomethacin or vehicle (V2) were administered to the different groups of mice at 8:00, 14:00 and 20:00.

At 09:00 on day 4 from the treatment with vehicle (V1) or NalO₃ the mice were sacrificed (according to the previously reported modalities) and the eyeballs were enucleated and processed for subsequent damage analysis.

### Example 3—Assessment of the Retinal Pigment Epithelium Damage

Damage to the RPE (retinal pigmented epithelium) was assessed by direct immunofluorescence. RPE corresponds to the pigmented cell layer just outside the neurosensory retina that nourishes the retinal visual cells, and is firmly attached to the underlying choroid and the overlying visual retinal cells.

The staining intensity of the RPE layer was quantified using a primary antibody (RPE65, #ab13826, mouse monoclonal, 1:100, Abcam, Cambridge, UK) to which a second antibody (fluorophore-labelled, Alexa Fluor 488, #A28175, Thermo Fisher Scientific) binds in the 5 groups of mice treated with V2/V1, V2/NalO₃, dipyrone/NalO₃, propyphenazone/NalO and indomethacin/NalO₃. The cell nuclei were visualized using the DAPI organic dye (#ab228549, Abcam, Cambridge, UK).

FIG. 1A shows representative images and cumulative data of the immunofluorescent staining of the RPE layer, performed using a primary antibody (RPE65), in the retina collected on day 4 in the 5 groups of mice treated with V2/V1, V2/NalO₃, dipyrone/NalO₃, propyphenazone/NalO₃ and indomethacin/NalO₃. Data are presented as mean±SEM. *p<0.05 vs veh; § p<0.05 vs NalO₃. Statistical analysis using one-way analysis of variance (ANOVA) test and Bonferroni test.

A reduction in the intensity of RPE layer staining of 48.0±2.9% (P<0.01 vs. V1/V2) was observed in mice that received NalO₃ injection. The treatment with dipyrone and propyphenazone eye drops statistically significantly reduced the damage induced by NalO₃ on the RPE layer compared to V2 by 87.8±7.8% (P<0.01) and 99.2±23.0% (P<0.01), respectively (FIGS. 1A and 1B). In contrast, indomethacin induced a modest and non-significant reduction of 5.7±14.9% in the staining intensity of the RPE layer (FIGS. 1A and 1 B).

The level of oxidative stress was also assessed throughout the thickness of the retina by measuring the immunofluorescence intensity for the reactive carbonyl species, 4-hydroxynonenal (4-HNE), a final indicator of oxidative stress.

### Example 4—Assessment of Oxidative Stress at the Retinal Level

Levels of 4-HNE were quantified using a primary antibody (#ab48506, monoclonal mouse [HNEJ-2], 1:40, Abcam, Cambridge, UK) to which a second antibody (fluorophore-labelled, Alexa Fluor 594, #A A32742, Thermo Fisher Scientific) binds in the 5 groups of mice treated with V2/V1, V2/NalO₃, dipyrone/NalO₃, propyphenazone/NalO₃ and indomethacin/NalO₃.

Cell nuclei were visualized using the DAPI organic dye (#ab228549, Abcam, Cambridge, UK).

The administration of NalO₃ induced an increase of 187.5±12.8% (P<0.001 vs. V1/V2) in 4-HNE immunofluorescence throughout the retinal tissue (FIGS. 2A and 2B).

While the treatment with dipyrone and propyphenazone markedly reduced retinal 4-HNE levels by 69.7±11.8% (P<0.001 vs. V2) and 81.3±7.0% (P<0.001 vs. V2) respectively, the treatment with indomethacin produced a modest non-statistically significant reduction in 4-HNE levels of 17.5±10.5% (FIGS. 2A and 2B). FIG. 2A shows representative images and cumulative immunofluorescence staining data of the oxidative stress biomarker, 4-hydroxynonenal (4-HNE) with primary antibody (#ab48506, monoclonal mouse [HNEJ-2], 1:40, Abcam, Cambridge, UK) to which a second antibody (fluorophore-labelled, Alexa Fluor 594, #A A32742, Thermo Fisher Scientific) binds in the 5 groups of mice treated with V2/V1, V2/NalO₃, dipyrone/NalO₃, propyphenazone/NalO₃ and indomethacin/NalO₃. Data are presented as mean±SEM. *p<0.05 vs V1/V2; § p<0.05 vs V2/NalO₃. Statistical analysis using one-way analysis of variance (ANOVA) test and Bonferroni test.

From the experimental evidence, it is possible to conclude that dipyrone and propyphenazone thus have a protective effect on the NalO₃-induced damage of the cells of the RPE layer, which is of fundamental importance for the maintenance of the photoreceptor function of the macula.

Furthermore, dipyrone and propyphenazone were observed to protect the retina from NalO₃-induced increase in 4-HNE. The COX inhibitor (indomethacin) proved unable to protect against RPE damage or NalO₃-induced increase in 4-HNE.

1: A method of preventing and/or treating a degenerative retinal disease which comprises administering to a patient in need thereof an effective amount of a compound Ceompeund belonging to the pyrazolone class of formula (I) 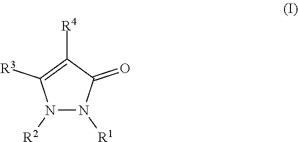
wherein:
R1 and R2 are independently selected from H, linear or branched C1-C6 alkyl, aryl optionally substituted with OH, C1-C6 alkoxy, linear or branched C1-C6 alkyl or halogen;
R3 is selected from linear or branched C1-C6 alkyl and OH;
R4 is selected from H, linear or branched C1-C8 alkyl, linear or branched C2-C8 alkenyl, a group -(CH₂)₁₋₄—CO-linear or branched C1-C6 alkyl, an amino group mono or disubstituted with linear or branched C1-C6 alkyl or with a group —(CH₂)₍₁₋₃₎SO₃H and combinations thereof, a —NHCO-aryl or —NHCO-heteroaryl group, and pharmaceutically acceptable salts thereof.

2: The method according to claim 1, wherein:
R1 and R2 are independently selected from H, linear or branched C1-C3 alkyl, phenyl optionally substituted with OH;
R3 is selected from linear or branched C1-C3 alkyl and OH;
R4 is selected from H, linear or branched C1-C6 alkyl, linear or branched C2-C6 alkenyl, a group -(CH₂)₍₂₋₃₎—CO-linear or branched C1-C4 alkyl, an amino group mono or disubstituted with linear or branched CT-C3 alkyl, with a group —(CH₂)₍₁₋₂₎—SO₃H and combinations thereof, a —NHCO-nitrogenated heteroaryl group, and pharmaceutically acceptable salts thereof.

3: The method according to claim 1, wherein the compound is selected from the group consisting of aminophenazone, dipyrone, phenazone, propyphenazone, nifenazone, phenylbutazone, pyrasanone, oxyphenylbutazone, kebuzone, feprazone, mofebutazone, tribuzone and mixtures thereof.

4: The method according to claim 1, wherein said degenerative retinal disease is selected from the group consisting of macular degeneration, diabetic retinopathy, retinal detachment, central serous chorioretinopathy, hypertensive retinopathy, macular hole, macular pucker, myodesopsia (floaters) and myopic maculopathy.

5-9. (canceled)

10: A method of preventing and/or treating a degenerative retinal disease which comprises administering to a patient in need thereof an effective amount of a pharmaceutical composition comprising at least one compound belonging to the pyrazolone class as defined in claim 1 and at least one pharmaceutically acceptable excipient.

11-13. (canceled)

14: The method according to claim 10, wherein said compound belonging to the pyrazolone class is selected from the group consisting of dipyrone, propyphenazone and mixtures thereof.

15-16. (canceled)

17: The method according to claim 10, wherein the pharmaceutical composition is an ophthalmic composition comprising the at least one compound belonging to the pyrazolone class, and at least one ophthalmologically acceptable excipient.

18: The method according to claim 17, wherein said ophthalmic composition is a topical ophthalmic composition, preferably an aqueous solution.

19: The method according to claim 10, wherein the compound belonging to the pyrazolone class is present in a concentration ranging from about 0.0001% to about 5% w/v.

20-21. (canceled)

22: A method of preventing and/or treating a degenerative retinal disease which comprises simultaneously, separately, or sequentially administering to a patient in need thereof an effective amount of a combination of at least one compound belonging to the pyrazolone class and an anti-VEGF drug and/or a corticosteroid drug.

23. (canceled)

24: The method according to claim 22, wherein the anti-VEGF drug is selected from the group consisting of ranibizumab, bevacizumab, and aflibercept.

25: The method according to claim 22, wherein corticosteroid drug is selected from the group consisting of cortisone, prednisone, prednisolone, methylprednisolone, meprednisone, beclomethasone, triamcinolone, paramethasone, mometasone, budesonide, fluocinonide, halcinonide, flumethasone, flunisolide, fluticasone, betamethasone, dexamethasone, hydrocortisone and fluocortolone.

26: The method according to claim 22, wherein the at least one compound belonging to the pyrazolone class is selected from the group consisting of dipyrone, propyphenazone and mixtures thereof.

27: A kit comprising a topical ophthalmic composition, a container containing it and a dispenser, wherein said topical ophthalmic composition comprises at least one compound belonging to the pyrazolone class and at least one ophthalmologically acceptable excipient.

28: A kit according to claim 27, wherein the topical ophthalmic composition is an aqueous solution.

29: A kit according to claim 27, wherein the at least one compound belonging to the pyrazolone class is selected from the group consisting of aminophenazone, dipyrone, phenazone, propyphenazone, nifenazone, phenylbutazone, pyrasanone, oxyphenylbutazone, kebuzone, feprazone, mofebutazone, tribuzone and mixtures thereof.

30: The method of claim 10, wherein the degenerative retinal disease is selected from the group consisting of macular degeneration, diabetic retinopathy, retinal detachment, central serous chorioretinopathy, hypertensive retinopathy, macular hole, macular pucker, myodesopsia (floaters) and myopic maculopathy.

31: The method according to claim 22, wherein the degenerative retinal disease is selected from the group consisting of macular degeneration, diabetic retinopathy, retinal detachment, central serous chorioretinopathy, hypertensive retinopathy, macular hole, macular pucker, myodesopsia (floaters) and myopic maculopathy.
